(19) United States
(12) Patent Application Publication
(10) Pub. No.: US 2003/0224492 A1
(43) Pub. Date: Dec. 4, 2003

(54) Method for site-directed mutagenesis

(76) Inventor: Lei Young, Sydney (AU)

Correspondence Address: Lei Young, 37/13 Waine St, Surry Hills, Sydney, 2010, AU

(21) Appl. No.: 10/249,809

(22) Filed: May 9, 2003

(30) Foreign Application Priority Data
May 29, 2002 (AU) PS2634

(51) Int. Cl. C12P 19/34; C12N 15/74

(52) U.S. Cl. 435/91.2; 435/488

(57) ABSTRACT

A method for site-directed mutagenesis which achieves mutant strand selection by introducing two flanking mutagenic primers.

### BACKGROUND OF INVENTION

The present invention concerns a method for performing site-directed mutagenesis used in genetic engineering techniques, more easily and efficiently, and a kit for the use in the above method.

Method of site-directed mutagenesis has evolved rapidly since the initial description of this concept [Smith, M. Annu Rev Genet. 19:423-462 (1985)], and has proved to be a remarkably useful tool in molecular biology. Polynucleotides having pre-determined sequences may now be designed at will. A common feature of the available methods is the use of synthetic oligonucleotides carrying the desired changes in the nucleotide sequence at the site of mutagenesis. This “mutant” oligonucleotide is incorporated into the sequence of interest by replacing the normal sequences with the designed oligonucleotide. This is accomplished by in vitro enzymatic DNA synthesis.

Conventionally, a method for performing site-directed mutagenesis comprises, for instance, the following procedures: (1) First, a desired DNA to be mutation-introduced is inserted to a vector. Thereafter, the complementary strand of the desired DNA is dissociated by heat or alkaline denaturation, in the case of using a double-stranded plasmid DNA, or a M13 phage vector is used to prepare a single-stranded DNA; (2) An oligonucleotide designed to introduce a desired mutation (mutagenic primer) is annealed with the above single-stranded DNA. Thereafter, a complimentary strand DNA is synthesized in vitro system by a reaction of DNA polymerase and DNA ligase; (3) Escherichia coli is transformed with the DNA obtained in the above item (2), and then a clone in which a mutation is introduced is selected.

However, with all currently described methods, the efficiencies of mutant selection are sufficient only for single-site mutagenesis, and the mutant to wild-type ratio drops dramatically with increased sites of mutation. Furthermore, most methods also require the DNA of interest to be cloned into a vector before mutagenesis can be carried out, adding to the already complicated procedures. Therefore, an object of the invention is to provide a simple and practical method for performing site-directed mutagenesis using PCR method, and a kit for carrying out the method for performing site-directed mutagenesis.

As a result of intensive investigation to develop an efficient and simple method for performing site-directed mutagenesis, the present inventor has developed a new technique for site-direct mutagenesis that have an advantageous combination of features as compared to other techniques for site-directed mutagenesis. These useful features include: (1) any DNA can be used as template, (2) consistently high mutation efficiency for any number of mutations, and (3) a minimal number of steps, thereby permitting the generation of host cells containing the mutant sequences in less than 24 hours.

### SUMMARY OF INVENTION

The present invention concerns a method for the incorporation of one or more specific mutations into a selected DNA molecule for mutagenesis, starting from a single-stranded DNA molecule, said method consisting the steps of:(a) annealing said single stranded DNA molecule simultaneously to two 5′-phosporylated mutagenic primers flanking the nucleotides to be mutated and one or more 5′-phosporylated mutagenic primers for the nucleotides to be mutated; and (b) extension of the annealed mutagenic primers by a DNA dependent DNA polymerase lacking 5′->3′ exonuclease activity, using said single stranded DNA molecule as template; and(c) ligation of the primer extension products by a DNA ligase when they are still annealed to said single-stranded DNA molecule; and (d) selective amplification of the ligated mutant strand by PCR with primers matching said ligated mutant strand, wherein the 3′ end of said PCR primers match the mutated nucleotides in the two flanking mutagenic primers in (a). The resulting PCR product can be cloned with any existing cloning method.

The present invention allows first strand cDNA to be used as template for mutagenesis, thus eliminating the requirement for cloning prior to mutagenesis. Furthermore, as the selection of mutants is achieved by PCR, which allows up to millions of folds of enrichment of mutant over wild-type clones, the mutagenesis efficiency is virtually 100%. The invention also allows optimal annealing of all mutagenic primers, resulting in highly efficient multiple site mutagenesis.

### BRIEF DESCRIPTION OF DRAWINGS

FIG. 1 This FIGURE provides a schematic diagram of an embodiment of the subject methods of site-directed mutagenesis.

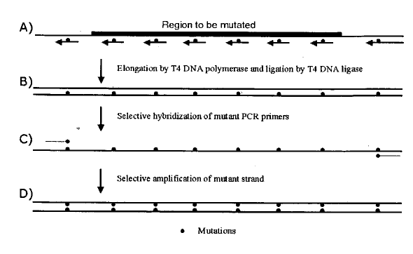

Single stranded DNA in Step (A) derived either from first-strand cDNA, M13 virus DNA or linear cyclic amplification of double stranded DNA, is annealed to two flanking anchor mutagenic primers outside the region to be mutated, which is indicated by a solid rectangle, together with one or more target mutagenic primers, the mutation sites are indicated by a solid circle. Step (B) shows the newly synthesized mutant strand hybridized with the wild type template strand, the mutant strand is selectively amplified in step (C), by the two PCR primers having their 3′ most nucleotides matching the anchor mutations, giving rise to a greatly enriched double stranded mutant DNA in step (D), which can be cloned by any available method.

### BRIEF DESCRIPTION OF SEQUENCES

Primer A1: 5′-CGCTCGTCGTCGATCACGGCTC-3′. 5′ flanking mutagenic primer

Primer A2: 5′-AAAACCTACTGTGCGCA-3′. 3′ flanking mutagenic primer

Primer P1: 5′-GCGCTCGTCGTCGATCA-3′. 5′ selection PCR primer

Primer P2: 5′-TTGTTTTCTGCGCACAG-3′. 3′ selection PCR primer

Primer M1: 5′-TGTACGTTGATATCCAG-3′. Base substitution introducing EcoRV

Primer M2: 5′-TCCTGCGTCTAGACCTG-3′. Base substitution introducing XbaI

Primer M3: 5-GCGCGGCTACAGCTCGAGCTGCCTGACG-3′. 120 bp deletion introducing XhoI

Primer M4: 5′-CTGGGCATGGATCCGTCCTGTGGCA-3′. 3 bp insertion introducing BamHI

Primer M5: 5′-AGCGCAAGTMTCCGTG-3′. Base substitution eliminating ScaI

### DETAILED DESCRIPTION

The present invention concerns, among other things, an improved method for site-directed mutagenesis. The improved methods of site-directed mutagenesis described herein provide for selection of the mutant strand by simple Polymerase Chain Reaction (PCR), and may be used to introduce one or more mutations in DNA sequences of interest.

The methods of the invention comprise the steps of annealing in addition to the mutagenic primers that correspond to the desired mutation positions, two flanking mutagenic primers both 5′ and 3′ to the desired sequence, to a single stranded DNA. Said single stranded DNA can be first strand cDNA with the complementary mRNA removed by Ribonuclease H, or prepared as part of a M13 phage vector single stranded DNA, or by linear cyclic extension of a single PCR primer, the template of which can be a plasmid, a PCR product of genomic DNA or cDNA. To allow optimal annealing of all primers, the annealing temperature needs to be gradually reduced from at least 75 Â° C. over 30-60 minutes period to less than 30 Â° C.

The mutagenic primers should be designed to contain at least 6 nucleotides 3′ to the mutated nucleotides, and at least 10 nucleotides 5′ to the mutated nucleotides. To allow the primers to anneal to the template before the formation of secondary structures in the template, the Tₘ of the mutagenic primers should be greater than the percentage GC content of the respective mutagenic primers, this can be adjusted by the length of the primers.

The concentration of each of the mutagenic primer is not particularly limited. However, it is desirable that the two flanking mutagenic primers are at lower concentration to the other mutagenic primers. Usually, as guidance, it is preferable that the flanking mutagenic primers are added at a concentration range of 50 nM to 200 nM, and all other mutagenic primers at 5 times that concentration. The 5′ terminals of the primers need to be phosphorylated, either by chemical synthesis, or by a kinase enzyme reaction such as the phage T4 polynucleotide kinase.

The synthesis of the mutant strand may be accomplished by the extension reaction from the annealed primers, catalysed by a polymerase enzyme lacking both 5′->3′ exonuclease activity, and strand displacement activity. Examples of such enzymes include, but are not limited to, phage T4 polymerase and phage T7 polymerase. The nicks that are formed in the mutant strand as a result of the primer extension can be sealed by a DNA ligase, such as the phage T4 DNA ligase.

In order to selectively amplify the mutant strand and not the parental wild-type strand, two additional selection PCR primers are designed, the 3′ end of which match the mutations incorporated into the mutant strand by the two flanking mutagenic primers. Primer extension from these two primers by a DNA polymerase allows selective replication of the mutant strand, as such reactions require the 3′ end of the primers to match the template sequence, which in this case is the mutant strand. In a preferred embodiment, the polymerase used is a thermostable polymerase. The polymerase used may be isolated from naturally occurring cells or may be produced by recombinant DNA technology. The use of Pfu DNA polymerase (Stratagene), a DNA polymerase naturally produced by the thermophilic archae Pyrococcus furiosus is particularly preferred for use in the PCR reaction steps of the claimed invention. Pfu DNA polymerase has been proved to have less error rate. Examples of other thermostable enzymes that may be used include, but are not limited to, Taq polymerase, Vent™ (New England Biolabs, Beverly Mass.) DNA polymerase, Deep Vent™ DNA polymerase (New England Biolabs, Beverly Mass.), and the like. In other embodiments of the invention, any DNA polymerase can be used for the selective amplification of the mutant strand. When the DNA molecule for mutagenesis is relatively long, it may be desirable to use a mixture of thermostable DNA polymerase, wherein one of the DNA polymerases has 5′-3′ exonuclease activity and the other DNA polymerase lacks 5′-3′ exonuclease activity. A description of how to amplify long regions of DNA using these polymerase mixtures can be found, among other places, in U.S. Pat. No. 5,436,149; Cheng, S et al. Proc. Natl. Aca. Sci. USA 91:5695-9 (1994); and Barnes, W M. Proc. Natl. Aca. Sci. USA 91:2216-2220 (1994). Use of a 3′->5′ exonuclease activity containing polymerase may degrade the 3′ mutated nucleotides of the PCR primers, to minimise this effect, the flanking mutagenic primers can be designed to contain at least 2 consecutive mutated nucleotides. The PCR reaction allows enrichment of the mutant strand by up to million fold, therefore allowing a mutagenesis efficiency of virtually 100%. The mutant PCR product may be cloned by any of the existing method, to facilitate cloning, the flanking mutagenic primers can be designed to create 2 new restriction sites, which can be cloned by simple restriction digestion and ligation into a vector. The 2 new restriction sites can also be used for the selection of the mutant strand. For example, after the synthesis of the first mutant strands, they can be separated from the wild-type template and re-annealed to the corresponding selection PCR primer, which initiates the synthesis of the second mutant strand. Only the DNA containing both mutant strands can be digested at the newly introduced restriction sites, therefore cloning with these restriction sites accomplishes selection of the mutant strand at the same time. If the single stranded template is prepared as part of a M13 phage vector DNA, which is synthesized in vivo and therefore is virtually free of unwanted mutations, and the synthesis of both mutant strands are through T4 DNA polymerase, which has the highest fidelity of all currently available polymerases, unwanted mutations will be kept to a minimum.

The kinds of mutations that can be introduced by the method for performing site-directed mutagenesis of the present invention are not particularly limited. Examples thereof include base substitution, deletion and insertion. Although the size of mutation is not subject to limitation, it is preferable that the length of the mutagenic primer is changed according to the size of mutation. For example, when the size of mutation is about 1 to 3 bases, the length of the mutagenic primer is preferably about 17-20 bases. When the size of mutation is 4 bases or more, it is preferable that a mutagenic primer of as long as about 30 bases is used. In addition, in the present invention, as to the number of the mutagenic primer, a desired mutation can be introduced by means of a single kind of mutagenic primer. When introducing different mutations at one site, however, a number of mutagenic primers depending on the purposes may be used in mixture to obtain a gene in which each of the desired mutations is introduced by a single operation. For example, when base-substitution site-directed mutagenesis for which alteration of a base at one site from G to A, T or C, respectively, is carried out, genes resulting from introducing mutation of from G to A, T or C, respectively, can be obtained by a single operation by preparing each three kinds of mutagenic primers, and then by using the mutagenic primers in mixture.

The cloned mutant product can be transformed into hosts for propagation. Methods for transforming hosts, such as Escherichia coli, with PCR product and other hosts include the calcium chloride method [Hanahan, D. J Mol Biol, 166:557-580 (1983)] and the electroporation method [Dower, W J et al. Nucleic Acids Res, 16:6127-6145 (1988)]. Regarding screening of the transformants obtained, direct PCR amplification of the clones with the final PCR primers will give rise to a band on an agarose gel only for the mutant clones.

The present invention is hereinafter described in more detail by means of the following examples, but the present invention is not limited to those examples.

### EXAMPLE 1

Two Single-Base Substitution Mutations.

1. Two mutagenic oligonucleotides complementary to the target mutations, and 2 flanking anchor mutagenic oligonucleotides containing 3 consecutive mutations were synthesized. Optionally, the oligonucleotides can be 5′ phosphorylated.

2. Single stranded template was prepared by linear cyclic amplification of a previously prepared plasmid, extending from a single primer in the strand complementary to all the mutagenic oligonucleotides. The resulting DNA should contain annealing sites for all the mutagenic oligonucleotides. The PCR conditions were 94 Â°0 C. for 2 minutes for 1 cycle; 94 Â° C. for 30 seconds, 56 Â° C. for 30 seconds, 72 Â° C. for 1 minutes/kb for 30 cycles; and finally 1 cycle at 72 Â° C. for 5 minutes.

3. Flanking anchor mutagenic oligonucleotides (100 nM) and target mutagenic oligonucleotides (500 nM) were mixed in 50 mM Tris-HCl (pH 7.5), 10 mM MgCl₂, 10 mM DTT, 1 mM ATP and 200 ÂμM dNTP.

If the oligonucleotides are not 5′ phosphorylated, 0.25 U/Âμl T4 polynucleotide kinase should be added, and the reaction tube incubated at 37 Â° C. for 30min. 10 ng/Âμl of the single stranded template was then added and the mixture incubated at 75 Â° C. with the temperature gradually decreased over a 30 min period to room temperature to allow complete annealing of the mutagenic primers. This can be accomplished by incubating the tube in 2 L of water which was initially heated to 75 Â° C.

4. Then 0.25 U/Âμl T4 DNA polymerase and 0.1 U/Âμl T4 DNA ligase were added to the mixture and incubated at 37 Â° C. for 30 min to allow synthesis of the mutant strand.

5. The mutant strand was selectively amplified by a thermostable DNA polymerase, with selection PCR primers having 3 nucleotides at the 3′ end matching the mutations introduced by the 2 flanking anchor mutagenic primers. PCR conditions were the same as step 2. The PCR product contains all the desired mutations and is ready for cloning.

### EXAMPLE 2

Control Reactions for the Proposed Mutagenesis Kit

1. The single stranded template DNA for the control reaction is the Â [symbol] actin first strand cDNA. cDNA synthesis was carried out with Superscript II reverse transcriptase following manufacturer's (Invitrogen) protocol in a 20 Âμl reaction, starting from 5 Âμg of total RNA. 2 U of ribonuclease H was then added and the reaction incubated at 37 Â° C. for 30 min to remove the template RNA.

2. Primers A1, A2, M1, M2, M3, M4 and M5 (1 Âμl each) as described in the “Brief description of sequences” section were mixed with 2 Âμl of cDNA synthesized in step 1, in final volume of 20 Âμl with other contents same as in example 1, step 3.

3. Follow the protocols in example 1 from step 3-5 with the exception of replacing the selection PCR primers in step 5 with Primers P1 and P2.

What is claimed is:

1. A method of introducing one or more specific mutations into a selected DNA molecule for mutagenesis, starting from a single-stranded DNA molecule, said method consisting the steps of:
(a) annealing said single stranded DNA molecule simultaneously to two 5′-phosporylated mutagenic primers flanking the nucleotides to be mutated and one or more 5′-phosporylated mutagenic primers for the nucleotides to be mutated; and
(b) extension of the annealed mutagenic primers by a DNA dependent DNA polymerase lacking 5′->3′ exonuclease activity, using said single stranded DNA molecule as template; and
(c) ligation of the primer extension products by a DNA ligase when they are still annealed to said single-stranded DNA molecule; and
(d) selective amplification of the ligated mutant strand by PCR with primers matching said ligated mutant strand, wherein the 3′ end of said PCR primers match the mutated nucleotides in the two flanking mutagenic primers in (a). The resulting PCR product can be cloned with any existing cloning method.

2. The method according to claim 1 wherein said single stranded DNA molecule is complementary DNA with the template RNA strand removed by ribonuclease H.

3. The method according to claim 1 wherein said single stranded DNA molecule is a circular phagemid DNA generated by f1 helper phage.

4. The method according to claim 1 wherein said single stranded DNA molecule is generated by means of a linear cyclic amplification reaction using a double stranded DNA molecule as template.

5. The method according to claim 4 wherein said double stranded DNA is generated by PCR amplification of genomic DNA.

6. The method according to claim 4 wherein said double stranded DNA is generated by PCR amplification of complementary DNA.

7. The method according to claim 4 wherein said double stranded DNA is plasmid DNA.

8. The method according to claim 4 wherein said double stranded DNA is cloned DNA.

9. The method according to claim 1 wherein the mutagenic primers contain at least 6 nucleotides 3′ to the mutated nucleotides, and at least 10 nucleotides 5′ to the mutated nucleotides.

10. The method according to claim 1 wherein the Tₘ of the mutagenic primers are greater than the percentage GC content of the respective mutagenic primers.

11. The method according to claim 1 wherein the flanking mutagenic primers contain at least 2 consecutive mutated nucleotides.

12. The method according to claim 1 wherein the flanking mutagenic primers create 2 new restriction sites for the facilitation of cloning and/or mutant selection.

13. The method according to claim 1 wherein the 5′ phosphorylation of the mutagenic primers is completed by a kinase, such as the T4 polynucleotide kinase.

14. The method according to claim 1 wherein the DNA polymerase in (b) is T4 DNA polymerase, T7 DNA polymerase or Klenow fragment of E.coli DNA polymerase I.

15. The method according to claim 1 wherein the phosphorylation, primer extension and ligation are performed in the same buffer.

16. The method according to claim 1 wherein the mutations introduced can be base substitutions, insertions and deletions.

17. A kit for introducing mutations into a selected DNA molecule for mutagenesis, said kit comprising: a polynucleotide kinase, a DNA polymerase lacking 5′->3′ exonuclease activity, a DNA ligase, a ribonuclease H, 5× mutagenic buffer and control mutagenic primers for a house keeping gene.

18. A kit according to claim 17, wherein said house keeping gene is Î²-actin.
